(19) United States
(12) Patent Application Publication
(10) Pub. No.: US 2017/0001029 A1
(43) Pub. Date: Jan. 5, 2017

(54) METHODS FOR CONTROLLING A MAGNETIC STIMULATION DEVICE

(71) Applicant: BTL HOLDINGS LIMITED, Nicosia (CY)

(72) Inventors: Ondrej Pribula, Praha (CZ); Tomás Franta, Dobrichovice (CZ); Jakub Ladman, Praha (CZ); Ondra Prouza, Ricany u Prahy (CZ)

(21) Appl. No.: 15/099,274

(22) Filed: Apr. 14, 2016

Related U.S. Application Data
(63) Continuation-in-part of application No. 15/073,318, filed on Mar. 17, 2016.
Application No. 15/073,318 is a continuation-in-part of application No. 14/951,093, filed on Nov. 24, 2015.
Application No. 14/951,093 is a continuation-in-part of application No. 14/926,365, filed on Oct. 29, 2015.
Application No. 14/926,365 is a continuation-in-part of application No. 14/789,658, filed on Jul. 1, 2015.
Application No. 14/789,658 is a continuation-in-part of application No. 14/873,110, filed on Oct. 1, 2015.
Application No. 14/873,110 is a continuation of application No. 14/789,156, filed on Jul. 1, 2015, now abandoned.

(51) Int. Cl. A61N 2/02; G05B 17/02; G06F 19/00

(52) U.S. Cl. CPC A61N 2/02; G06F 19/3406; G05B 17/02

(57) ABSTRACT

Methods of calculating and/or suggesting the maximal value of stimulation parameter are based on the safety concept of a magnetic stimulation device for treatment of a biological structure by a high power time-varying magnetic field. The safety concept protects the patient. The methods may be used e.g. in physiotherapy, psychotherapy, psychiatry or aesthetic medicine applications.

### PRIORITY CLAIM

This application is a Continuation-in-Part of each of the following U.S. Patent Applications: Ser. No. 15/073,318 filed Mar. 24, 2015 and now pending; Ser. No. 14/951,093 filed Nov. 24, 2015 and now pending; Ser. No. 14/926,365 filed Oct. 29, 2015 and now pending; and Ser. No. 14/789,658 filed Jul. 1, 2015 and now pending. This application is also a Continuation-in-Part of U.S. patent application Ser. No. 14/873,110 filed Oct. 1, 2015 and now pending, which is a Continuation of U.S. patent application Ser. No. 14/789,156 filed Jul. 1, 2015 and now abandoned. Each of these applications is incorporated herein by reference.

### FIELD OF THE INVENTION

The present invention relates to methods of protecting a patient from unintended heat generation during the treatment by a time-varying magnetic field. The safety concept may be evaluated statistically and/or calculated based on the parameters set up by an operator.

### BACKGROUND OF THE INVENTION

Presently, stimulation for treatment and improvement of patient's well-being and/or appearance is performed by magnet treatment methods reaching low repetition rates or direct current methods. In the case of more intensive treatments the magnetic stimulation device operation is limited by the factory settings and/or by the worst potential cases which are predefined by the dependence of the maximal values of magnetic flux densities and the repetition rates. The treatment is limited by the preset operation parameters.

Following the state of art the stimulation by a time-varying magnetic field is limited by key parameters of repetition rate, magnetic flux density and/or treatment duration. The applicator may exceed a predetermined temperature which may cause heat injury to the patient where the treatment of the biological structure requires high magnetic flux density. If the magnetic stimulation device produces more heat than the cooling system can dissipate, the treatment device is turned off based on feedback from a temperature sensor in the applicator.

In commercially available magnetic stimulation devices the limit may be set by the operator. However, the control system of the magnetic stimulation device may operate the magnetic stimulation device to reach the predetermined magnetic flux density which is determined empirically by the manufacturer during the most discriminating stimulation, e.g. the case of the highest repetition rate. Therefore the stimulation is limited in the magnetic flux density domain and/or the depth of the stimulated target tissue is limited. Furthermore, the stimulation is also limited by the repetition rate and/or the treatment duration.

Additionally, no commercially available magnetic stimulation device is able to monitor the stimulation energy and protect the patient and/or the magnetic stimulation device from an unintended event by monitoring the current value of the operation parameter. Therefore, an unintended event may cause heat damage to the patient and/or the magnetic stimulation device without ceasing the treatment. Unintended events may cause the operation parameter drop so the efficiency of the magnetic stimulation device decreases if an unintended event occurs. Power consumption may increase during the unchanged treatment parameters, placing the patient and/or the magnetic stimulation device at risk.

### SUMMARY OF THE INVENTION

The present invention provides a new approach in determining the parameters of biological structure treatment.

According to the first aspect of the invention the magnetic stimulation device monitors the stimulation energy based on the current value of an operation parameter and/or the operation parameter waveform of one period.

According to another aspect of the invention the magnetic stimulation device may include additional thermal protection based on mathematic and/or signal processing methods which determine the relation of the currently determined operation parameter with a reference and/or with the operation parameter measured in a different value of characteristic quantity.

According to still another aspect of the invention the magnetic stimulation device may determine, based on the transition thermal characteristic of the magnetic stimulation device, the maximal treatment parameters which can be sufficiently cooled by the cooling system.

According to still another aspect of the invention the control unit may calculate optimal flow of the cooling medium based on the treatment parameters, transition thermal characteristic of the magnetic stimulation device and/or the cooling medium temperature and significantly reduce the noise of the cooling system.

According to still another aspect of the invention the control unit may optimize the treatment parameters based on the current value of operation parameters and a transition thermal characteristic of the magnetic stimulation device.

### BRIEF DESCRIPTION OF THE DRAWINGS

FIG. 1 illustrates a voltage calibration curve of one impulse measured in the time domain.

FIGS. 2A-2G illustrate a difference of a voltage calibration curve in the case of a metal object in proximity of the magnetic stimulation device.

FIG. 3 illustrates a difference of voltage calibration curve in the case of a hardware error of the magnetic stimulation device.

FIG. 4 illustrates a diagram of a calculation algorithm operating with a plurality of inputs.

FIG. 5 illustrates an exemplary application of the calculation algorithm.

### LIST OF REFERENCE NUMBERS

1 voltage calibration curve (full line)

2 measured voltage waveform (dashed line)

3 second maximum of measured voltage (incorrect)

4 second maximum of calibration curve (correct)

5 time shift

6 voltage drop (incorrect)

7 first maximum

8 voltage drop (correct)

9 resonance effect

10 calibration voltage value at same time

10′ currently measured voltage value at same time

11 calibration voltage value at different time

11′ currently measured voltage value at different time

12′ currently measured voltage value at time t1

13′ currently measured voltage value at time t1+x

14 calculation algorithm

15 transition thermal characteristic

16 real energy losses

17 treatment parameters

18 actual temperature of the magnetic stimulation device

19 cooling parameters

20 result

21 start of magnetic stimulation device

22 input parameters

23 determine T_{proc}

24 comparing T_{Dmax} with Tₘₐₓ

25 disable treatment

26 calculate and suggest at least one maximal treatment parameter

27 start of treatment

28 measure T_{M}(t)

29 differing T_{M}(t) with T_{D}(t)

30 continue treatment

31 end

32 comparing T_{M}(t) with T_{D}(t)

33 comparing T_{M}(t) with Tₘₐₓ

34 disable treatment

### Glossary

Patient refers to any living organism, such as human or animal.

Stimulation refers to a magnetic flux density inducing an electric current in the biological structure.

Biological structure includes a cell, a neuron, a nerve, a muscle fiber, a tissue, a filament or an organ.

Magnetic stimulation device refers to a complete magnetic stimulation device or any part of it, such as an applicator, a stimulating coil, resistors, wires etc.

Calibration curve refers to the representative waveform of an operation parameter determined by mathematic and/or signal processing method, i.e. it refers to a plurality of values of an operation parameter determined in different values of characteristic quantity.

Calibration value refers to a correct value of an operation parameter which is established by factory settings, mathematical model, mathematic and/or signal processing methods.

Operation parameter impulse refers to an operation parameter waveform inducing one impulse.

Impulse refers to a single magnetic stimulus.

Pulse refers to a period of stimulation by a time-varying magnetic field of at least one magnetic stimulus and a time duration of no stimulation, i.e. time duration between two impulses from rise/fall edge to next rise/fall edge.

Repetition rate refers to the frequency of pulses; it is derived from the time duration of a pulse.

Operation parameter refers to voltage, current or magnetic flux density.

Currently determined value of the operation parameter refers to the value of the operation parameter determined at a specified time during the currently examined magnetic pulse.

Characteristic quantity refers to time, frequency, amplitude or phase.

Treatment parameters refer to magnetic flux density, repetition rate, impulse duration or treatment duration.

Input parameters refer to treatment parameters, real and/or theoretical energy losses, transition thermal characteristic, actual temperature of the magnetic stimulation device, ambient temperature or cooling medium temperature and/or flow.

Mathematical model refers to an abstract model using mathematical language to describe the thermal behavior of a magnetic stimulation device.

Mathematic method refers to a calculation and/or statistic method.

Statistic method refers to any statistic quantity, e.g. mean, modus, median, running average, correlation and/or correlation coefficient.

Signal processing method refers to any method of signal processing, e.g. Fourier transformation, wavelet transformation, filtering etc.

Reference refers to the calibration curve and/or to the reference value measured in the same value of the characteristic quantity.

Normalized conditions refer to predetermined properties of the operation parameter, e.g. period, measurement time etc.

Energy storage device refers to a capacitor or other electrical energy storage device which is charged by a power supply and discharged to provide a current flow creating the magnetic field.

To relate refers to any relation of at least two values of operation parameter, i.e. relation may be correlation, correlation coefficient, ratio or any other method expressing the similarity of at least two values by mathematic and/or signal processing method.

### DETAILED DESCRIPTION OF THE INVENTION

Based on the state of art there is great insufficiency in the field of magnetic stimulation treatment specifically, overheating a magnetic stimulation device, or insufficient and/or even a very inaccurate treatment planning. Current commercially available magnetic stimulation devices determine a temperature of the applicator measured by a temperature sensor. If the temperature reaches a predetermined temperature then the treatment is stopped until the temperature falls below the predetermined temperature to enable a continuation of the treatment.

There is also no magnetic stimulation device which is able to generate a notification for operating personnel in case any unintended event occurs and/or which is able to distinguish from at least two unintended events.

An important parameter for the safe concept is the temperature of the magnetic stimulation device. Heat is generated by total energy losses which are caused by static and dynamic components. The static energy loss component is represented by ohmic resistance described by Equation 1

$${E}_{R} = ρ \cdot \frac{l}{S} \cdot {I}^{2} \cdot {t}_{imp} \qquad Eq . 1$$

where: E_{R} is the energy loss (J); ρ is the resistance (Ω·m); l is the length of wire (m); S is the surface area (m²); I is the current (A); tᵢₘₚ is the time of an impulse (s).

The dynamic energy loss component is represented by energy loss generated by eddy currents described by Equation 2

$${E}_{EDDY} = \frac{{π}^{2} \cdot {B}_{P}^{2} \cdot {d}^{2} \cdot {f}^{2}}{6 \cdot k \cdot ρ \cdot D} \cdot m \cdot {t}_{imp} \qquad Eq . 2$$

where: E_{EDDY} is energy loss (J); Bₚ is the peak of magnetic field (T); f is frequency (Hz); d is the thickness of the sheet or diameter of the wire (m); k is constant equal to 1 for a thin sheet and 2 for a thin wire; p is the resistivity of material (Ω·m); D is the density of material (kg·m³); m is weight of wire material; tᵢₘₚ is the time of an impulse(s).

Therefore the total energy loss is given by Equation 3.

E_{TOT}=ΣEᵢ=E_{EDDY}+E_{R}, Eq. 3

where: E_{TOT} is the total energy loss (J); E_{EDDY} is the energy loss of eddy currents (J); E_{R} is the energy loss of ohmic resistance (J).

The power losses generate heat which increases the temperature of the magnetic stimulation device. The heat is distributed within the magnetic stimulation device and the heat is dissipated by the cooling medium flow.

The generated heat needs to be monitored since overheating may cause damage to the magnetic stimulation device and heat damage of the medical device may also be a potential risk for the patient. Therefore heat generation has to be monitored to prevent thermal damage to the magnetic stimulation device and/or the patient.

Power losses and/or heat generation may be monitored and/or determined by the magnetic stimulation device based on determining the waveform of any operation parameter, e.g. voltage, electric current or magnetic flux density. The determined waveform is related with a reference and/or with the operation parameter measured in a different value of a characteristic quantity, e.g. time, frequency, amplitude or phase.

According to the invention a current value of an operation parameter, e.g. voltage, electric current or magnetic flux density, may be determined by measuring via a suitable sensor or by deriving from a value of voltage source, e.g. an energy storage device or power source. The currently determined operation parameter is processed by a mathematic and/or signal processing method.

According to one application of the invention the at least one currently determined operation parameter may be used for determining a correctness of the stimulation. The correctness of the stimulation may be determined by the relation between a current value of an operation parameter and a reference or the operation parameter measured in a different value of characteristic quantity. The relation is result of a mathematic and/or signal processing method.

According to one aspect of the application a calibration curve may be established. The calibration curve is calibration waveform of the operation parameter. The calibration curve may be implemented by the manufacturer as a factory setting. Alternatively, the calibration curve may be established by a mathematic and/or signal processing method. The calibration curve may be determined from at least one waveform, more preferably at least 2 waveforms, even more preferably at least 5 waveforms, even more preferably 10 waveforms, most preferably at least 50 waveforms. The reference may be established by the complete calibration curve, a representative segment of the calibration curve or by predefined reference points of the calibration curve, e.g. a look-up-table.

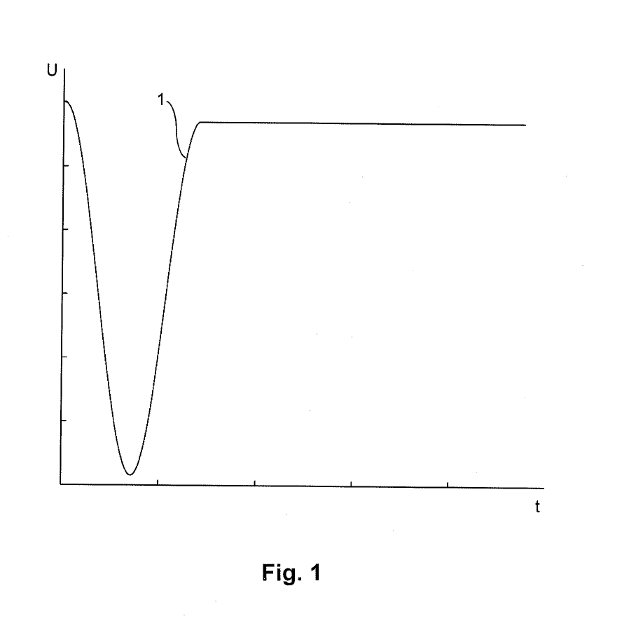

FIG. 1 illustrates a voltage calibration curve 1 of one impulse measured in the time domain. The voltage waveform may be determined e.g. on an energy storage device. However any operation parameter may be used for establishing the calibration curve.

The currently measured voltage waveform and the calibration curve are related using a mathematic and/or signal processing method. Based on the relation at least one threshold may be established. The at least one threshold may correspond to the correctness of the stimulation and/or notify operator of the magnetic stimulation device about an unintended event. The unintended event may refer to detection of a metal object e.g. metal jewelry such as ring or bracelet, or a prosthetic device such as an endoprosthesis or surgical nail within the proximity of the magnetic stimulation device; or to detection of a hardware error of the magnetic stimulation device, e.g. error of the switching device such as a thyristor. Based on the evaluation of any unintended event the treatment may be disabled and/or the notification for the operator may be generated by the magnetic stimulation device in a human perceptible form, e.g. by mechanical and/or electromagnetic means, such as audibly perceptible notification (e.g. beep) or visually perceptible notification (flashing light, color change etc.).

In an exemplary application of the aspect of the application, the relation between currently measured voltage waveform and the voltage calibration curve may be determined by a statistic method resulting in a correlation coefficient. The time duration of the correlated calibration curve and the voltage waveform may be longer than the time duration sufficient to reach the value of a second maximum. The correct stimulation may be determined if the correlation coefficient value is in absolute value at least 0.9, more preferably at least 0.95, most preferably at least 0.99. The unintended event may be detected if the correlation coefficient value is in absolute value at least 0.4, more preferably at least 0.6, even more preferably at least 0.7, most preferably at least 0.9. The value of correlation coefficient may be used for detection of a metal object within the proximity of the magnetic stimulation device e.g. metal jewelry such as a ring or bracelet, or a prosthetic device such as an endoprosthesis or a surgical nail; or for detection of hardware error of the magnetic stimulation device, e.g. error of a switching device such as thyristor.

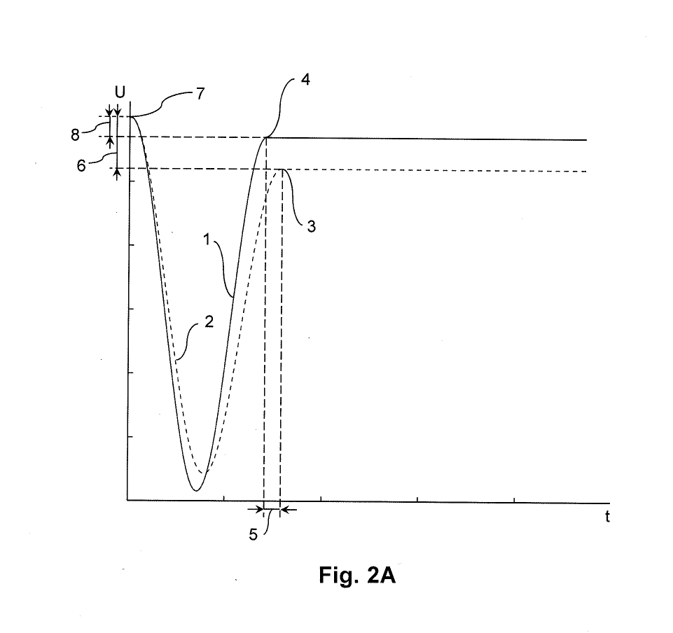

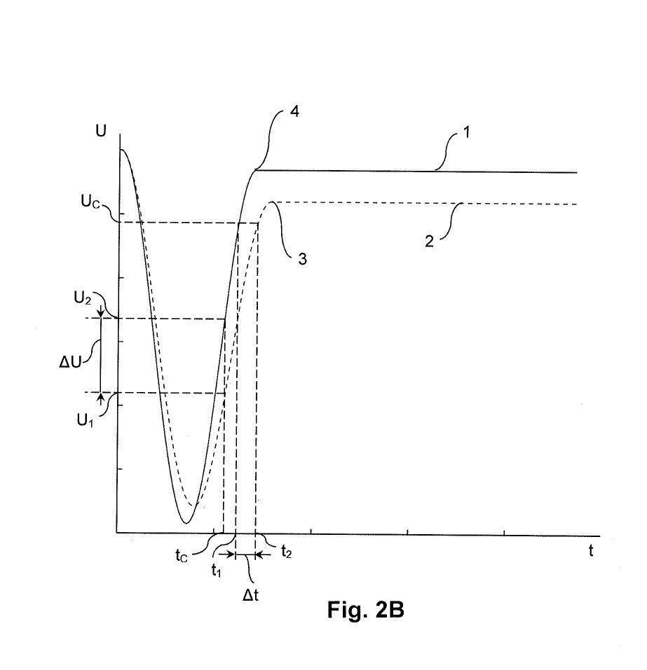

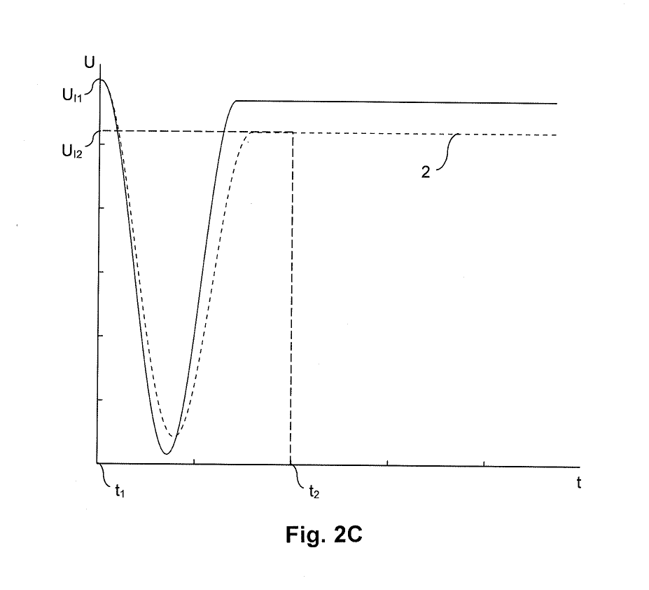

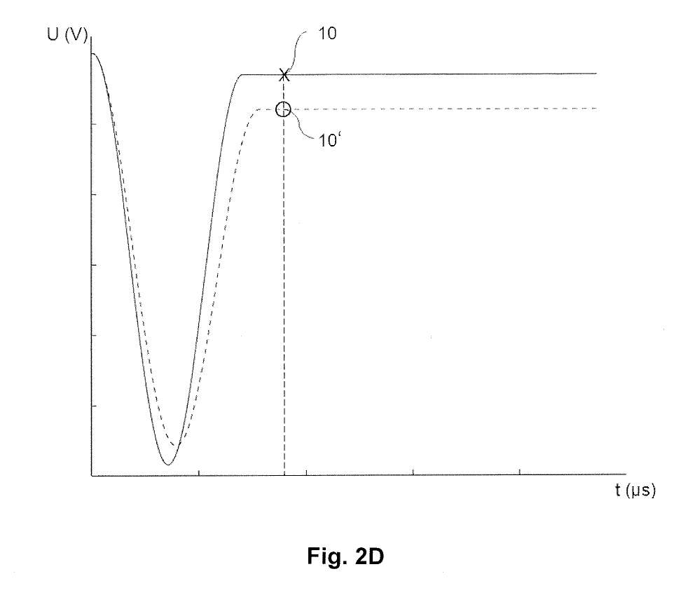

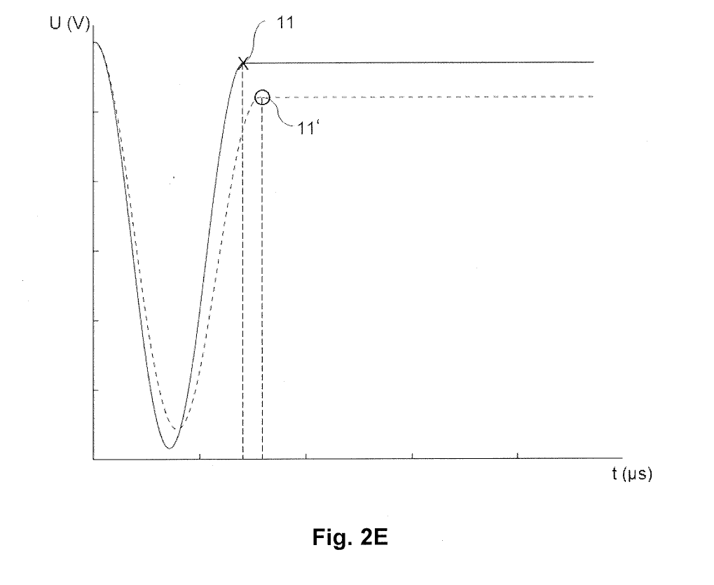

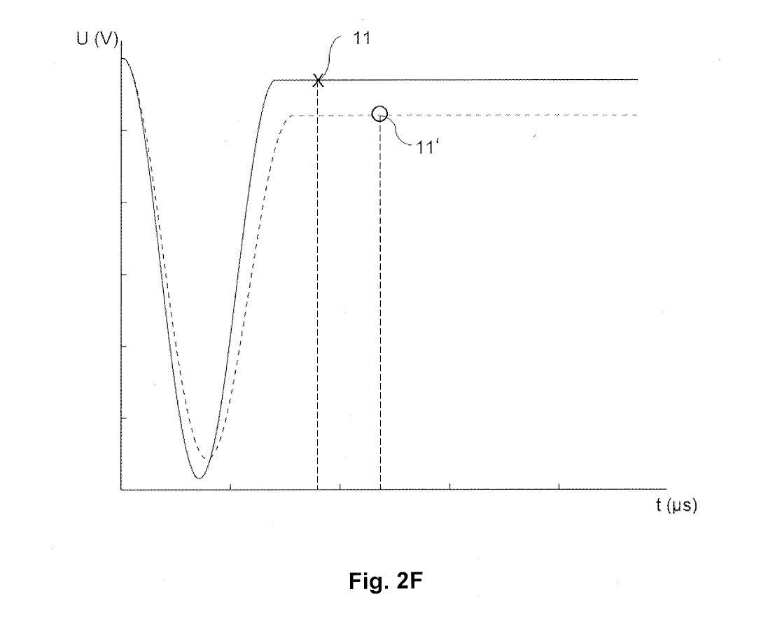

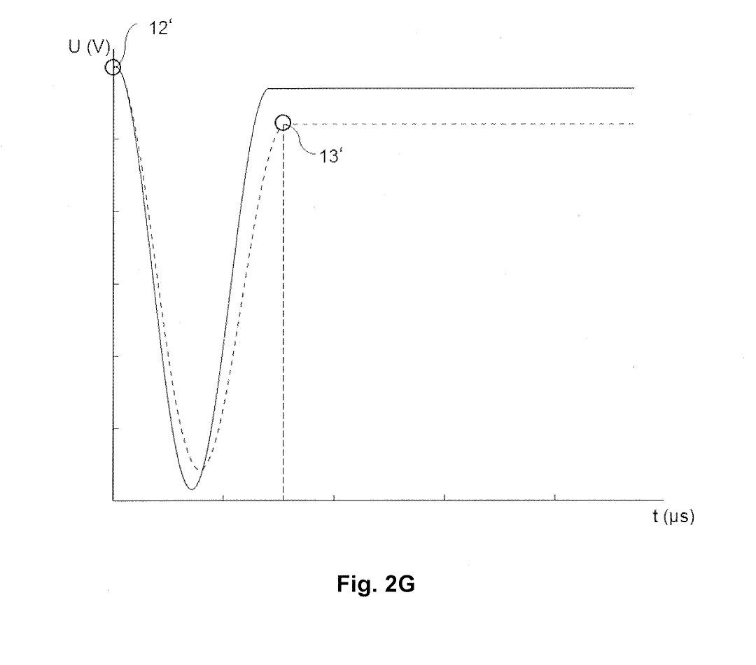

FIG. 2A illustrates the case when the metal object is within proximity of the magnetic stimulation device. There are two curves which refer to a voltage calibration curve 1 and the currently measured voltage waveform 2. The currently measured voltage waveform 2 differs in the value of second maximum 3 compared to the value of second maximum 4 of the voltage calibration curve 1. Further additional difference occurs in time shift 5 referring to the time when the currently measured voltage reaches the value of second maximum 3 compared to the time when the calibration curve reaches the value of second maximum 4. Therefore the correlation coefficient may reach lower values in absolute values than in the case of correct stimulation when the value of the correlation coefficient in absolute value is at least 0.9, more preferably at least 0.95, most preferably at least 0.99. The detection of a metal object is very important for the patient's safety due to risk of injury caused to the patient by heat induction in the metal object and/or by the unintended movement of the metal object.

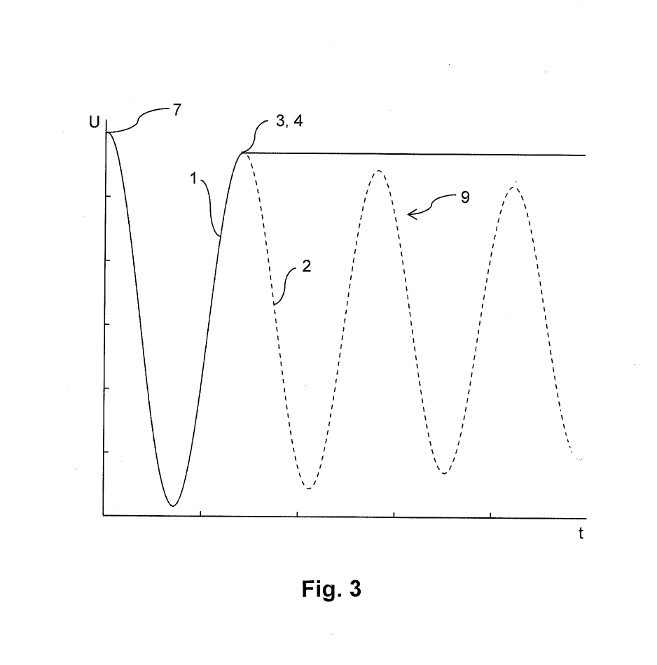

FIG. 3 illustrates the case when a hardware error occurs, e.g. a failure of the switching device. There are two curves which refer to a voltage calibration curve 1 and the currently measured voltage waveform 2. The voltage calibration curve 1 value remains constant after reaching the value of second maximum 4. However, the currently measured voltage waveform 2 continues in resonance 9 although the value of second maximum 3 equals to the value of second maximum 4 of the calibration curve 1.

In the preferred application the relation between the voltage calibration curve 1 and the currently measured voltage waveform 2 may be determined by a time period longer than time duration sufficient to reach the value of second maximum of the operation parameter.

The calibration curve may be set by the manufacturer or by a mathematic and/or signal processing method. The magnetic stimulation device may verify and/or adjust the calibration values periodically after a predetermined time period and/or after changing any part of the magnetic stimulation device.

The benefit of using the correlation coefficient is that the method is independent of repetition rate and/or amplitude of the stimulation. The method also provides very precise and/or relevant results.

In an alternative aspect of the application, the correctness of the treatment may be determined simply by a relation of the at least one specific value of the currently measured voltage waveform 2 influenced by the metal object. The metal object may absorb a part of the stimulation energy. Therefore the currently measured voltage is lower than the calibration value and the currently determined voltage drop 6 is increased as is illustrated in FIG. 2A. The value of first maximum 7 corresponds with the maximum stimulation voltage generated by a voltage source, it may be simply determined from the voltage source. During the correct treatment based on energy losses a recharge of the energy storage device is not up to the value of first maximum 7 but only up to the value of second maximum 4 which is less than the value of first maximum 7. Therefore a correct voltage drop 8 occurs which is determined by the difference of the value of first maximum 7 and the value of second maximum 4. The correct voltage drop 8 corresponds with the value of first maximum 7. The voltage drop occurs within each impulse. Therefore a threshold of correct voltage drop may be set up. In the case of no unintended event and the correct treatment, the value of currently measured voltage corresponds with the calibration value and the correct voltage drop 8 remains constant during the constant operation parameters and/or ambient conditions. With respect to correct voltage drop 8 a predetermined voltage drop threshold may be set up which corresponds with the correct magnetic stimulation and which may be considered as being correct. The correct values may be calibrated by the manufacturer or may be determined by mathematic and/or signal processing methods. The magnetic stimulation device may verify and/or adjust the calibration values periodically after a predetermined time period and/or after changing any part of the magnetic stimulation device.

The correct voltage drop threshold may be established at 30%, more preferably 21%, even more preferably 14%, most preferably 7% of the value of first voltage maximum. If the voltage drop reaches the threshold then the proximity of the metal object may be determined. If the voltage drop varies in time after reaching the value of second maximum then a hardware error may be detected. The notification relating to an unintended event may be generated in human perceptible form.

In an alternative approach, the correctness of the treatment may be also determined only by the relation values of second voltage maximums 3, 4 and/or by the relation of any other reference points in voltage calibration curve 1 and the currently measured voltage waveform 2. The reference points and/or threshold may be established by the manufacturer as factory settings, e.g. a look-up-table, or the reference points and/or threshold may be established by the operator.

During the treatment several cases may occur. In the exemplary application the operation parameter may be voltage. These cases are:

1) The correct stimulation case where the currently measured voltage (a specific value or a waveform) is identical or within an acceptable range of a reference or the voltage value measured in different time of the same pulse (correlation coefficient equals almost 1).

2) The incorrect stimulation case, which may be determined by the relation of:

a) The currently measured voltage waveform and the calibration curve; or

b) The currently measured value of voltage measured at a predetermined time t and the predetermined correct value of voltage at time t. (e.g. the time t may be the moment of reaching the second maximum). If the relation exceeds a predefined threshold then an incorrect stimulation case is present and the magnetic stimulation device generates a notification to the personnel.

c) The currently generated voltage is measured in two different times: time t and time t+x and the currently measured values of voltage are related together. If the relation exceeds a predefined threshold then an unintended event is present and the magnetic stimulation device generates a notification to the personnel.

As shown in FIG. 2B, the correctness of the stimulation may be determined by at least one reference point in the currently measured voltage waveform. In the preferred application the value of second maximum is used because it is well defined and it may be easily determined. On the other side, the correctness of the stimulation may be determined by the relation of at least two reference points. One exemplary application may determine a voltage difference ΔU=U₂−U₁ at time t_{c}. Based on the value of the voltage difference proximity of metal object may be determined. In this exemplary application U₂ is constant because it is derived from a calibration value. Another exemplary application may determine a time difference Δt=t₂−t₁ from when a calibration value and the measured voltage reach a selected voltage U_{c}. Then based on the value of the time difference proximity of metal object may be determined. In this exemplary application t₁ is constant because it is derived from a calibration value.

FIG. 2C shows determining an incorrect stimulation by currently measured values of voltage (U_{I2}, U_{I2}) measured in predetermined values of time (t₁, t₂). The correctness of the stimulation may be determined by the relation of U_{I1}(t₁) and U_{I2}(t₂). If the relation exceeds a predefined threshold then an unintended event is present and the magnetic stimulation device generates a notification to the personnel. In the preferred application the values of first and second maximum may be used.

In one aspect, a method of controlling a magnetic stimulation device for treating a biological structure by time-varying magnetic field includes determining at least one value, for example (Volts), of an operation parameter (Voltage) in at least one value (microseconds) of characteristic quantity (time), wherein the value of operation parameter is related to at least one of: a calibration curve; a calibration value; or at least one value (two voltage measurements at specific times) of the same operation parameter in a different value (microseconds) of the same characteristic quantity (time), wherein the calibration curve and/or the calibration value may be determined in the same value (microseconds) or in a different value (microseconds) of the same characteristic quantity (time). The calibration curve is plurality of calibration values (Volts in this example) of an operation parameter (Voltage) in a plurality of values (microseconds) of a characteristic quantity (time). A calibration value, in this example, is a specified voltage at a specified time.

In FIGS. 2D-2G, all currently measured values are shown as a circle with reference numbers marked with an apostrophe; all calibration values are marked as cross and reference numbers are without an apostrophe; all relations determined at the same time are reference numbers 10; and all relations determined at different times are reference numbers 11.

A complete waveform of one impulse is measured. The impulse (when the voltage value changes in time) lasts e.g. 280 μs during correct stimulation. The complete voltage waveform is related (using the definition in the glossary above) to the calibration curve (stored in memory of the magnetic stimulation device). The relation is expressed by the value of a correlation coefficient which indicates the similarity of the currently measured waveform and calibration curve.

Referring to FIG. 2D, the calibration voltage waveform 10 may be related to the currently measured voltage waveform 10′ with the same time duration, e.g. 350 μs, i.e. the time duration of calibration voltage waveform 10 equals the time duration of currently measured voltage waveform 10′. The complete voltage waveform need not be determined. It is sufficient to set at least one calibration value. The currently measured voltage value 10′ is related to the predetermined calibration voltage value 10. The ratio of the voltage values 10, 10′ (or value of voltage drop, or correlation) determines an incorrect stimulation. The currently measured voltage value 10′ and the calibration voltage value 10 may be determined at the same time.

Referring to FIG. 2E, the calibration voltage waveform 11 may be related to the currently measured voltage waveform 11′ with a different time duration. The currently measured voltage value 11′ is measured at the time when the second maximum is reached. The measured voltage second maximum occurs at a time different than the time of second maximum of calibration curve.

Turning to FIG. 2F, the currently measured voltage value 11′ is related to the predetermined calibration voltage value 11. The ratio of the voltage values 11, 11′ (or value of voltage drop, or correlation) determines an incorrect stimulation. The currently measured voltage value 11 and the calibration voltage value 11′ may be determined at different times. The result of the relation is the same, although the currently measured voltage 11′ is measured at a different time (at time 600 μs) than the calibration voltage value 11 (at time 400 μs).

Moving to FIG. 2G, the complete voltage waveform need not be determined, and any calibration voltage value does not have to be set. A relation between the currently measured voltage values 12′ and 13′ is determined. The at least two currently measured voltage values 12′, 13′ in the current pulse are measured at different times of the same pulse. The system may determine an incorrect stimulation e.g. based on knowledge of the correct voltage drop. The correct voltage drop may be determined by the system manufacturer/operator as an absolute voltage value in Volts (dependent on a first maximum value); or by a ratio of currently measured voltage values with respect to a first maximum value; or a percentage of a first maximum value; or it may be derived from a mathematical model.

In an alternative application the magnetic stimulation device may send a notification concerning the hardware error to the service department and/or manufacturer to repair the device. The magnetic stimulation device may also include a black box for storing data concerning unintended events to provide a statistics for the operator and/or the manufacturer.

The benefit of the application is determining an unintended event within each impulse. Hence patient's safety is significantly improved and the patient and/or the magnetic stimulation device is prevented from heat damage. Additionally, the magnetic stimulation device may be able to provide a notification concerning the unintended event to the operating personnel in human perceptible form. Further benefit is recognizing the type of unintended event.

The application is not limited by the recited values and/or the recited characteristic quantities. Similar results may be achieved by using the current waveform/calibration curve and/or magnetic flux density waveform/calibration curve determined on the coil.

In one embodiment, a method of controlling a magnetic stimulation device for treating a biological structure by a time-varying magnetic field includes measuring a voltage of the device over a time interval; relating the measured voltage to a calibration curve; and turning the device off and/or providing a notification to the operating personnel, based on the relating of the measured voltage to the calibration curve. The method may further include determining a correlation coefficient between the measured voltage and the calibration curve; and turning the device off and/or providing a notification to the operating personnel, if the correlation coefficient is below a predetermined value.

In another method of controlling a magnetic stimulation device for treating a biological structure by a time-varying magnetic field, steps include: measuring a voltage of the device at a time T1; relating (i.e., comparing or otherwise determining a function of) the measured voltage at time T1 to a predetermined calibration voltage at time T1; or relating the measured voltage at time T1 to a predetermined calibration voltage at time T1+x; and then turning the device off and/or providing a notification to the operating personnel, based on the relating of the measured output voltage to the predetermined calibration voltage.

Alternatively, a method for detecting incorrect operation of a magnetic stimulation device for treating a biological structure by a time-varying magnetic field includes:

AA.] Determining that a relation between a measured voltage of the device and a calibration curve exceeds a predetermined threshold; or

BB.] Determining that a voltage measured at a predetermined time T1 and a correct voltage value of a calibration curve at time T1 exceeds a predetermined threshold; or

CC.] A relation of a first voltage measured at time T1 and a second voltage measured at time T1+x exceeds a predefined threshold.

The device is turned off, and/or a notification is provided to the operating personnel, based on the relating of the measured output voltage to the predetermined calibration voltage.

According to another application of the invention at least one currently determined operation parameter may be used for determining a value of the generated heat. The generated heat may be used for prediction of a temperature of the magnetic stimulation device. Typically the method may be used for treatment planning and/or to predict the temperature of the applicator and/or the part of the magnetic stimulation device which is the most susceptible to overheating such as wires and/or resistors etc.

The magnetic stimulation device may be described by a transition thermal characteristic (TTC). The TTC may be determined by experimental measurement during standard ambient conditions such as temperature and/or pressure, or it may be a mathematical model based on technical and/or electric specifications of all components of the magnetic stimulation device. TTC characterizes the temperature dependence of the magnetic stimulation device on heat. TTC is established by the manufacturer as a factory setting.

The value of generated heat determined by the recited application of the invention corresponds with the treatment parameters. The temperature evolution of the magnetic stimulation device is dependent during the treatment on at least one of treatment parameters, actual temperature of the magnetic stimulation device, ambient temperature, cooling medium temperature, cooling medium flow or heat dissipation.

A calculation algorithm is set up to operate at least TTC and treatment parameters to determine the temperature of the magnetic stimulation device during the treatment. The maximal temperature of the magnetic stimulation device is limited and predetermined. However, in alternative applications the maximal temperature of the magnetic stimulation device may be adjusted by the operator. The maximal temperature may be considered to be safe for the patient.

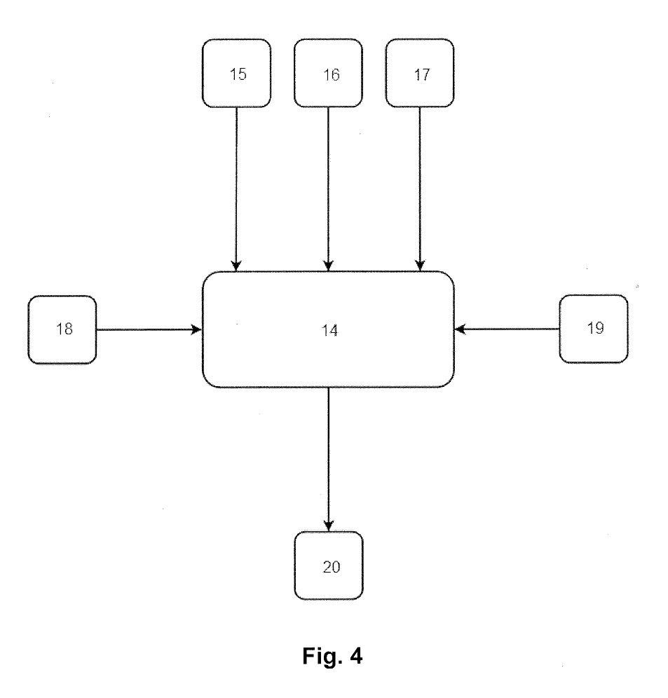

FIG. 4 illustrates a diagram of the calculation algorithm 14 operating with a plurality of inputs. Inputs may include TTC 15; real and/or theoretical energy loss 16 (e.g. from TTC); at least one treatment parameter 17 such as repetition rate, magnetic flux density, impulse duration, amplitude modulation and/or treatment duration; actual temperature 18 of the magnetic stimulation device; cooling parameters 19 such as ambient temperature, cooling medium temperature, flow and/or pressure gradient, relative humidity, heat capacity and/or heat dissipation. Based on the input parameters the other parameters concerning the treatment may be determined as a result 20. In the preferred application the real energy loss for the at least one pulse may be used.

According to one aspect of the application, the magnetic stimulation device may stop the treatment in the case that the temperature determined by the calculation algorithm exceeds the maximal temperature. If the calculated temperature equals the maximal predetermined temperature then the treatment is started since the maximal predetermined temperature is considered to be safe for the patient. The treatment is stopped only if the calculated temperature exceeds the maximal predetermined temperature.

According to another aspect of the application, the magnetic stimulation device may disable the treatment in the case that the temperature determined by the calculation algorithm exceeds the maximal temperature. In this case the magnetic stimulation device may suggest at least one maximal value of treatment parameter. Based on the predicted temperature of the magnetic stimulation device the calculation algorithm may determine at least one value of treatment parameter to not exceed the maximal temperature of the magnetic stimulation device during the treatment. Based on the operator's preferences the value of the treatment parameter may be automatically adjusted by the magnetic stimulation device or it may be suggested to the operator in human perceptible form such as audibly perceptible notification (e.g. beep) and/or visually perceptible notification (e.g. flashing light, color change etc.). In an exemplary application the suggested treatment parameter may be a maximal achievable value of magnetic flux density which can be sufficiently cooled by the cooling system. However, any other treatment parameter may be suggested to the operator.

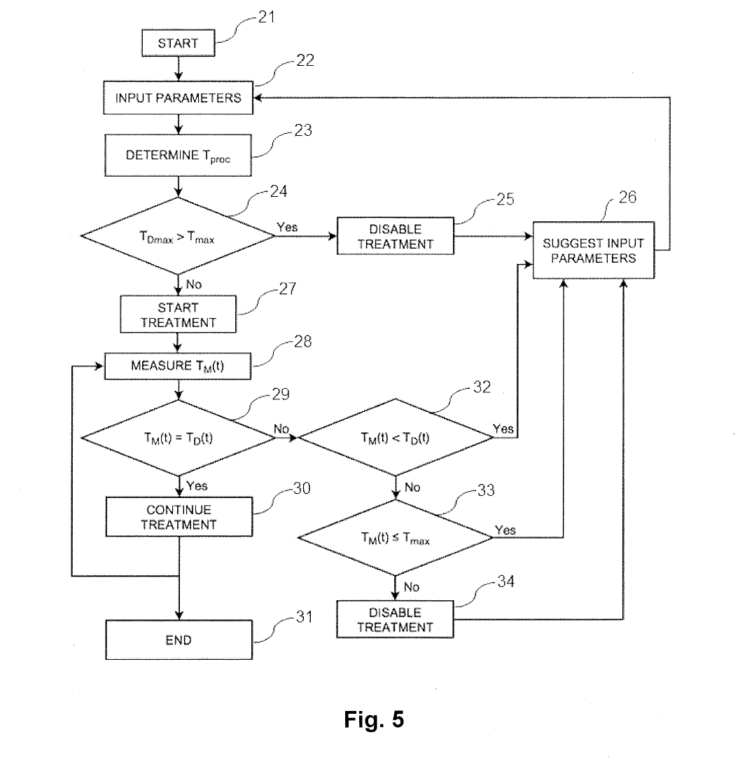

FIG. 5 illustrates a calculation algorithm to determine a maximal magnetic flux density which may be sufficiently cooled by the cooling system. As soon as the magnetic stimulation device is turned on 21 the operator may set the input parameters 22 which are considered by the operator as suitable for the patient. Next step 23 may follow. In the step 23, based on the input parameters the calculation algorithm may determine temperature distribution T_{proc} including at least one of a temperature of the magnetic stimulation device determined in time t of the treatment (T_{D}(t)) and the maximal temperature of the magnetic stimulation device (T_{Dmax}) which may be reached during the treatment. In the next step 24, the magnetic stimulation device may determine whether the determined maximal temperature of the magnetic stimulation device exceeds maximal predetermined temperature (Tₘₐₓ).

In the case that T_{Dmax} exceeds Tₘₐₓ, in step 25 the treatment may be disabled and/or a notification concerning the reason may be generated by the magnetic stimulation device in a human perceptible form. In the next step 26, the calculation algorithm may determine at least one maximal treatment parameter which may be reached to sufficiently cool the magnetic stimulation device and the magnetic stimulation device may suggest at least one maximal treatment parameter to the operator. Consequently, the operator may input 22 corrected treatment parameters within the acceptable cooling range.

If the magnetic stimulation device determines in the step 24 that T_{Dmax} doesn't exceed Tₘₐₓ, then the treatment may be started 27. Afterwards, the actual temperature of the magnetic stimulation device (T_{M}(t)) may be measured in step 28. The temperature measurement may be achieved in real time continuously or in discrete time sequences, more preferably in predetermined discrete time values.

In step 29 the magnetic stimulation device may determine whether T_{M}(t) differs from the determined temperature of the magnetic stimulation device (T_{D}(t)). If T_{D}(t) equals to T_{M}(t) then the treatment continues 30 by generating further magnetic impulse and by measuring the further T_{M}(t) until the end 27 of the treatment and/or until the block 29 examines the difference in T_{D}(t) and T_{M}(t).

In the case that T_{D}(t) and T_{M}(t) differs in step 29 in consequence step 32 may follow. In step 32 the magnetic stimulation device may examine whether the T_{M}(t) is lower than T_{D}(t). If T_{M}(t) is lower than T_{D}(t) then the calculation algorithm may determine at least one maximal treatment parameter which may be reached to sufficiently cool the magnetic stimulation device and suggest in step 26 the at least one new maximal treatment parameter to the operator who may adjust the at least one treatment parameter in step 22. The at least one new treatment parameter may be higher than the at least one originally suggested treatment parameter.

In the case that T_{M}(t) is not lower than T₀(t) then the magnetic stimulation device may examine in step 33 whether T_{M}(t) is lower than or equal to Tₘₐₓ. If T_{M}(t) is lower than Tₘₐₓ then the calculation algorithm may determine at least one maximal treatment parameter which may be reached in to sufficiently cool the magnetic stimulation device and the magnetic stimulation device may suggest in step 26 the at least one maximal treatment parameter to the operator who may adjust the at least one treatment parameter in step 22. The at least one new treatment parameter may be lower than the at least one originally suggested treatment parameter. If T_{M}(t) equals to Tₘₐₓ then the magnetic stimulation device may generate the notification that the maximal predetermined temperature was reached.

If the magnetic stimulation device examines in step 33 that T_{M}(t) is not lower than or equal to Tₘₐₓ then the treatment is disabled 34 since the temperature has exceed Tₘₐₓ and/or a notification may be generated by the magnetic stimulation device in a human perceptible form. The calculation algorithm may determine at least one maximal treatment parameter which may be reached to sufficiently cool the magnetic stimulation device and suggest in step 26 the at least one maximal treatment parameter to the operator who may adjust the at least one treatment parameter in step 22.

According to still another aspect of the application, the calculation algorithm may monitor the actual temperature of any part of the magnetic stimulation device, e.g. temperature of the applicator. The temperature of the applicator may be determined by real heat losses during the treatment without needing a temperature sensor in the applicator.

According to still another aspect of the application, the calculation algorithm may determine the cooling parameters, e.g. cooling medium flow, sufficient to cool the heat generated during the treatment. The consumption of the cooling may be optimized.

The calculation algorithm may run in real time. Accordingly, the application may monitor the actual temperature of the magnetic stimulation device and examine whether the treatment corresponds with the result based on determination by the calculation algorithm. The notification in human perceptible form may be generated and/or the treatment may be limited and the at least one treatment parameter may be suggested in human perceptible form, and/or the treatment may be disabled if the difference reaches a predetermined and/or adjustable threshold.

The methods described above may be utilized in any characteristic quantity domain.

The application method is not limited by the recited characteristic quantities. It should be interpreted in the broadest sense.

The application is not limited to the recited operation parameters and/or the characteristic quantity. Any combination of suitable operation parameters and/or characteristic quantities may be used as well. The method should be interpreted in the broadest sense.

The method is not limited to application of a magnetic field hence it may be also used for other equivalent treatment, e.g. radiofrequency treatment, light treatment and/or mechanical treatment such as ultrasound treatment or shock-wave treatment.

1. A method of controlling a magnetic stimulation device for treating a biological structure by time-varying magnetic field including:
determining at least one value of operation parameter in at least one value of a characteristic quantity,
wherein the value of operation parameter is related with at least one of:
a) a calibration curve,
b) a calibration value,
c) at least one value of the same operation parameter in the different value of the same characteristic quantity,
wherein the calibration curve and/or the calibration value may be determined in the same value or in the different value of the same characteristic quantity.

2. The method of claim 1 wherein the relation is determined by mathematic and/or signal processing method.

3. The method of claim 2 wherein the at least one treatment parameter is limited and/or the treatment is disabled if the relation exceeds a predetermined threshold.

4. The method of claim 2 wherein the at least one value of relation is established by factory settings and/or by an operator.

5. The method of claim 2 wherein the value of relation determines an unintended event.

6. The method of claim 5 wherein the unintended event is a metal object within proximity of the magnetic stimulation device and/or a hardware error of the magnetic stimulation device.

7. The method of claim 3 wherein the limited treatment parameter and/or disabled treatment protects the patient and/or the magnetic stimulation device from heat damage.

8. The method of claim 3 wherein the limited treatment parameter and/or disabled treatment protects the patient from injury caused by movement of the a metal object.

9. The method of claim 5 wherein the unintended event is notified in a human perceptible form.

10. The method of claim 8 wherein the notification in human perceptible form is generated by audible and/or visual means.

11. The method of claim 1 wherein the calibration curve is established as factory settings.

12. The method of claim 1 wherein the calibration curve is determined by mathematic and/or signal processing method of the at least one operation parameter waveform.

13. The method of claim 12 wherein the calibration curve is determined from at least ten waveforms.

14. The method of claim 1 wherein the calibration curve and/or calibration value is established as factory settings in value of operation parameter measured during standard ambient conditions.

15. The method of claim 2 wherein the relation is determined by at least one predetermined point of a currently measured waveform.

16. The method of claim 15 wherein the at least one predetermined point is set as the factory settings.

17. The method of claim 15 wherein the at least one predetermined point is set by the operator.

18. The method of claim 15 wherein the predetermined points are first and second maximum of the measured waveform.

19. The method of claim 2 wherein the relation corresponds to heat generation.

20. The method of claim 19 wherein the actual heat is determined by mathematic and/or signal processing method.

21. The method of claim 19 wherein the actual heat is determined by relation of the value of operation parameter in first and second maximums values of the measured waveform.

22. The method of claim 2 wherein a coil is operated to maximally reach a predetermined temperature of the magnetic stimulation device.

23. The method of claim 22 wherein a transition thermal characteristic of the magnetic stimulation device is determined.

24. The method of claim 23 wherein the transition thermal characteristic is a thermal function of the coil.

25. The method of claim 22 wherein the at least one treatment parameter is adjustable by the operator and wherein the at least one treatment parameter may be suggested by the magnetic stimulation device to the operator.

26. The method of claim 25 wherein the magnetic stimulation device determines the at least one maximal treatment parameter based on actual temperature of the applicator and/or the at least one of the other treatment parameters.

27. The method of claim 22 wherein the maximal predetermined temperature of the magnetic stimulation device is adjustable by the operator.

28. The method of claim 27 wherein the magnetic stimulation device operates the cooling medium flow to sufficiently cool the applicator and/or the coil to reach the adjusted and/or predetermined temperature.

29. The method of claim 22 wherein an actual temperature of the applicator and/or the coil is determined by the at least one of actual ambient air temperature, cooling medium temperature, magnetic flux density, repetition rate, impulse duration or treatment duration.

30. The method of claim 19 wherein the heat is determined by the relation of the calibration curve and currently measured operation parameter waveform.
